(19) United States
(12) Patent Application Publication
(10) Pub. No.: US 2009/0016979 A1
(43) Pub. Date: Jan. 15, 2009

(54) ALUMINUM AND ALUMINUM-ZIRCONIUM COMPOSITIONS OF ENHANCED EFFICACY CONTAINING REDUCED BUFFER AND/OR REDUCED ZIRCONIUM

(76) Inventors: Zijun Li, Westfield, NJ (US); Mark Rerek, Scotch Plains, NJ (US)

Correspondence Address: ARTHUR J. PLANTAMURA, ESQ.;GENERAL CHEMICAL PERFORMANCE PRODUCTS LLC., 90 EAST HALSEY ROAD, PARSIPPANY, NJ, 07054, US

(21) Appl. No.: 11/762,455

(22) Filed: Jul. 9, 2007

(51) Int. Cl. A61K 8/28

(52) U.S. Cl. 424/66; 424/68

(57) ABSTRACT

Basic aluminum chloride (BAC) and aluminum zirconium antiperspirant active of enhanced efficacy are proposed. The acid activated aluminum-zirconium composition having low contents of both glycine and zirconium with Band III/II ration of less than 0.5 is found to provide higher efficacy than the corresponding heat activated aluminum zirconium antiperspirant salt with high HPLC Band III/II ratio and having high contents of both glycine and zirconium at similar M/Cl ratio.

According to present invention, the interactions between amino acid such as glycine and aluminum species in BAC and aluminum-zirconium solutions greatly reduce the effective amount of aluminum monomer species, which attributed to the reduced efficacy and stability of the corresponding solutions. The aluminum and aluminum-zirconium antiperspirant salt compositions of enhanced efficacy have low molar ratio of amino acid/or buffer to metal(s) of 0.2 and preferably less than 0.15, with metal(s) to chloride molar ratio of at least 1.1. Preferably the aluminum-zirconium salts have aluminum to zirconium atomic ratio of 6 to 10. The compositions are further activated by acid, such as aluminum chloride.

### FIELD OF THE INVENTION

The present invention relates to enhanced efficacy aluminum and aluminum-zirconium antiperspirant actives characterized by relatively low proportions of both amino acid or other buffer and with reference to aluminum-zirconium antiperspirants, reduced zirconium that are also activated by a suitable acid.

This invention deals with the interactions between aluminum species and amino acid or other buffer used in basic aluminum chloride (BAC) and in aluminum-zirconium solutions and its effect on the efficacy and stability of the solutions, as well as on the development of other aluminum and aluminum-zirconium antiperspirant compositions of enhanced efficacy that comprise basic aluminum chlorides and aluminum-zirconium complexes with low amino acid or other buffer. The aluminum and aluminum-zirconium salts of the present invention have low molar ratio of amino acid or other buffer to Al or (Al+Zr) of about 0.05 to about 0.15 with high HPLC Band IV peak area of greater than 20% and metal(s) to chloride molar ratio greater than 1.1. The aluminum and aluminum-zirconium antiperspirant salts are activated by a suitable acid such as, hydrochloric acid and aluminum chloride.

### BACKGROUND OF THE INVENTION

Antiperspirants of enhanced efficacy generally have high HPLC Band III, with Band III to Band II area ratio of at least 0.5, and can be prepared by the dilution and heat treatment of aluminum chlorohydrate (ACH) solution referred as heat activation. U.S. Pat. No. 4,359,456; GB2048229A; U.S. Pat. Nos. 4,944,933; 5,356,609; 5,358,694 and U.S. Publication No. 2004/0101500 A1 describe the preparation of heat activated ACH salts i.e. AACH. Activated aluminum-zirconium antiperspirant salts are produced by the addition of zirconium compounds to the activated aluminum solutions. Similar to AACH, the resultant aluminum-zirconium solutions have to be dried quickly to powders due to the fast revert back of Band III to Band II of the activated solutions that results in the lose of the efficacy of the corresponding solutions. Patents describing the preparation of activated aluminum-zirconium antiperspirant salts include U.S. Pat. Nos. 4,775,528; 4,871,525; 4,871,525; 4,900,534; EU0653,203; U.S. Pat. Nos. 6,375,937 B1 and 6,436,381 B1. Aluminum or aluminum-zirconium antiperspirant salts prepared through heat activation are usually in powder forms and are not economical because of the lengthy heating time and drying of the diluted solution.

High efficacy aluminum antiperspirants can also be obtained through acid activation by addition of acid such as hydrochloric acid and aluminum chloride to basic aluminum chloride (BAC) solutions. The acid activated aluminum salts usually have low Al/Cl ratio of less than 1.8 and are characterized by having low HPLC Band I peak area of less than 5% and high HPLC Band IV peak area of greater than 20%. The corresponding aluminum-zirconium salts activated by acid can be prepared by adding zirconium complex to the acid activated BAC solutions. Different from heat activation, acid activated, aluminum or aluminum-zirconium salts are stable at higher salt concentration and are available in both solution and powder forms. Such acid activated salts have both economical and practical advantages over the heat activated actives. U.S. Pat. No. 6,902,724 B1 describes BAC compositions with Al/Cl ratio of 1.2 to 1.5 and preferably 1.3 to 1.4. it is realized that at lower Al/Cl ratio of 1.3 to 1.4, the aluminum salts can be irritant and may cause can corrosion when used as an aerosol. The patent mentioned that an amino acid such as glycine may be introduced to reduce the irritation. It does not mention the effect of amino acid on the efficacy and stability of the BAC solutions.

Publication No. WO 2004/052325 discloses a zirconium-free aluminum dichlorohydrate salt of high efficacy and low irritation with a nitrogen containing buffer, such as glycine or betaine, to aluminum molar ratio in the range of 0.05 to 0.26. In the examples., e.g., Examples 13 through 18, reductions in efficacy of aluminum dichlorohydrate solutions were observed at higher glycine content. Aluminum dichlorohydrate was used in the examples.

U.S. Pat. No. 4,331,609 reveals an efficacious Al—Zr tetra salt composition prepared through the acid activation by the addition of hydrochloric acid or aluminum chloride to the active solution. The composition has metal to chloride (M/Cl) molar ratio of less than 1.3 at high zirconium content, with Al/Zr ratio being 2.5 to 4.5. All the examples demonstrated in the patent have an Al/Zr ratio of 3.3. It does not mention the effect of glycine on the efficacy or stability of the Al—Zr salt compositions.

U.S. Pat. No. 6,375,937 B1 describes aluminum-zirconium salts having a M/Cl ratio of 0.9 to 1.2 and glycine/zirconium ratio of greater than 1.3. The higher ratio of glycine/zirconium is not economical.

U.S. Pat. No. 6,436,381 B1 discloses enhanced efficacy aluminum-zirconium tetra salts having M/Cl ratio of 0.9 to 1.0 with HPLC peak 5 of at least 33% and amino acid/zirconium ratio of about 1. Such low M/Cl ratio antiperspirant salts suffer from the problem of skin irritation and are difficult to formulate.

Int'l. Publication No. WO 03/094880 teaches an anhydrous topical antiperspirant composition comprising an aluminum-zirconium chlorohydrate with low M/Cl ratio of 0.9 to 1.3 and a stabilizing basic material that is separated from the antiperspirant salt. The presence of the basic material is sufficient to prevent the degradation of the antiperspirant composition and minimize skin irritation. The stabilizing basic material can be selected 1) the salts formed by a strong base and a week acid such as sodium carbonate; 2) amino acid that are basic in aqueous solution such as glycine; 3) salts formed by a strong base and an amino acid such as sodium glycinate; 4) inorganic base that are not soluble in water such as alkaline earth, oxides. None of the stabilizing material mentioned in the patent is relevant to the present invention.

U.S. Publication No. 2005/0214234 discloses novel antiperspirant compounds comprising basic aluminum halides that have a particular molecular size distribution, i.e. less than 5% HPLC Band I and Band III/II ratio of less than 0.5 in admixture with a metal salt, such as salts of titanium, hafnium, tin or zirconium, preferable zirconium, which is buffered with an amino acid such as glycine. The spirit of the invention is to include the zirconium compounds in combination with the efficacious BAC solutions to provide the corresponding aluminum-zirconium antiperspirant. The two examples illustrated by the application are aluminum-zirconium tetra salts with glycine/Zr molar ratio of about 1. The composition is outside the present invention.

The introduction of zirconium species to the aluminum salt depolymerizes the higher molecular weight aluminum species to lower molecular weight aluminum species together with the formation of aluminum dimer and monomer species. Such changes in aluminum structures and fester hydrolysis rates for zirconium complexes result in an increased efficacy. It is generally accepted that the presence of higher amount of zirconium complex further increases the efficacy of the aluminum-zirconium antiperspirant salts. Further, the known commercial antiperspirant salts claiming to have strong antiperspirancy and/or deodorancy are those containing high zirconium content salts such as aluminum-zirconium-tetra-/or -trichlorohydrex glycine actives with Al/Zr ratio of less than 6.0 and mostly less than 4. Because zirconium species hydrolyze and precipitate at relative lower pH than aluminum salts, amino acid such as glycine is added in order to form a stable zirconium solution at higher solution pH. Higher glycine is thus required for the aluminum-zirconium salts containing higher zirconium. About 40% to 50% of glycine is considered completed in aluminum-zirconium-glycine solution (A H Rosenberg; J J Fitzgerald, Chemistry of Aluminum-Zirconium-Glycine (AZG) Complexes, Antiperspirants and Deodorants 5:137-166, 1999) The effect of glycine (including both free and bounded) on the efficacy of the corresponding aluminum and aluminum-zirconium antiperspirant salt is uncertain.

The understanding of interaction between amino acid/or other buffer and aluminum species in aluminum and aluminum-zirconium antiperspirant salts and its effect on solution stability and efficacy is highly desirable.

None of the above prior art references disclose or teach acid activated antiperspirant compositions with reduced amino acid or other buffer and with reference to aluminum-zirconium antiperspirants, reduced zirconium. Such compositions demonstrate enhanced efficacy; the solutions are stable and they are economical as well.

### SUMMARY OF THE INVENTION

It is an object of the present invention to provide aluminum and aluminum-zirconium compositions of enhanced efficacy containing low amino acid/or buffer.

It is another object of the present invention to provide the aluminum-zirconium compositions of enhanced efficacy containing both low amino acid/or buffer and zirconium.

It is a further objective of the present invention to provide stable and economical acid activated aluminum and aluminum-zirconium antiperspirant salts with low amino acid/or buffer and/or low zirconium and that are also skin friendly.

In accordance with the present invention acid activated aluminum-zirconium compositions at low contents of glycine and zirconium with Band III/II ratio of less than 0.5 are found to provide higher efficacy than the corresponding heat activated aluminum-zirconium antiperspirant salts with high HPLC Band III/II ratio and having higher contents of glycine and zirconium at similar M/Cl ratio.

Pursuant to the invention, an aluminum-zirconium antiperspirant salt is prepared by mixing a 30% to 50% BAC solution with Al/Cl ratio below 1.8 having HPLC Band I of less than 5%, a zirconium hydroxy chloride solution and an amino acid/or buffer solution with amino acid such as glycine below 3% by weight in at least 30% (USP) solution. An acid agent such as, hydrochloric acid and aluminum chloride is added during preparation. Preferably the composition has an Al/Zr ratio of 8 or higher with M/Cl ratio of greater than 1.1. An 30% to 50% BAC solution with Al/Cl ratio of about 1.3 to about 1.4 can be buffered with amino acid/or buffer at molar ratio of amino acid/or buffer to Al of about 0.05 to about 0.15. Further the composition can be formulated in gel, roll-on, stick and cream and the aluminum only active can be formulated in aerosol also.

The enhanced efficacy of aluminum-zirconium antiperspirant salts derived from the low M/Cl ratio, especially between 0.9 and 1, has been attributed to the presence of higher HPLC Band IV (otherwise designated as peak 5). These salts, however are highly irritant to the skin because of the presence of higher amount of free acid. Both aluminum monomers and dimers are eluted as peak IV by HPLC size exclusion column, i.e. the size exclusion column does not differentiate Al monomer and dimers, which however, can be identified by ²⁷Al NMR, with Al monomers having a sharp signal at ˜0 ppm and the dimers showing a peak near 4-5 ppm. AlCl₃ solution shows only Band IV by SEC-HPLC and one sharp peak at 0 ppm by ²Al NMR. The introduction of zirconium species to the aluminum salt further depolymerizes the aluminum species as demonstrated by the increase in SEC-HPLC % Band IV and Al monomers and dimers at 0 ppm and 4-5 ppm by ²⁷Al NMR. The aluminum and aluminum-zirconium salts containing higher amount of aluminum monomers are regarded as more efficient in sweat reduction.

It is recognized that the introduction of the amino acid in the aluminum-zirconium antiperspirant salts not only increases the stability of the solutions by preventing the solutions from gelling through the coordination of amino acid with zirconium, but also buffer the solutions and keep the solutions from being too acidic. However, the inclusion of relatively high levels of zirconium and amino acid detract from the stability of the corresponding aluminum-zirconium solution because of the higher salt contents. Additionally the product with higher zirconium and/or high amino acid is less economical. Complete information regarding interactions between aluminum salts and amino acid such as glycine is relatively unavailable and would be important to determine that such interactions exist since about 50% free glycine is present in AZG solutions. According to the present invention we have discovered that amino acid or buffer such as glycine and betaine interacts with aluminum species in BAC solutions especially at lower M/Cl ratio. The interaction has been characterized by ²⁷Al NMR, which evidenced a reduction in peak intensity of Al monomers at 0 ppm, and without, any significant change in HPLC Band IV. The interaction between glycine and aluminum monomers reduces the effective amount of aluminum monomer species, results in the gelling of the BAC glycine and aluminum-zirconium glycine solutions especially at lower M/Cl ratio. Further the amount of aluminum monomers is increased by lowering the amount of amino acid or buffer and by the addition of aluminum chloride.

### BRIEF DESCRIPTION OF THE FIGURES

FIG. 1 is the ICP-SEC-HPLC of acid activated aluminum-zirconium-glycine powder at glycine/(Al+Zr) ratio of 0.10 according to experiment number 26 herein below described.

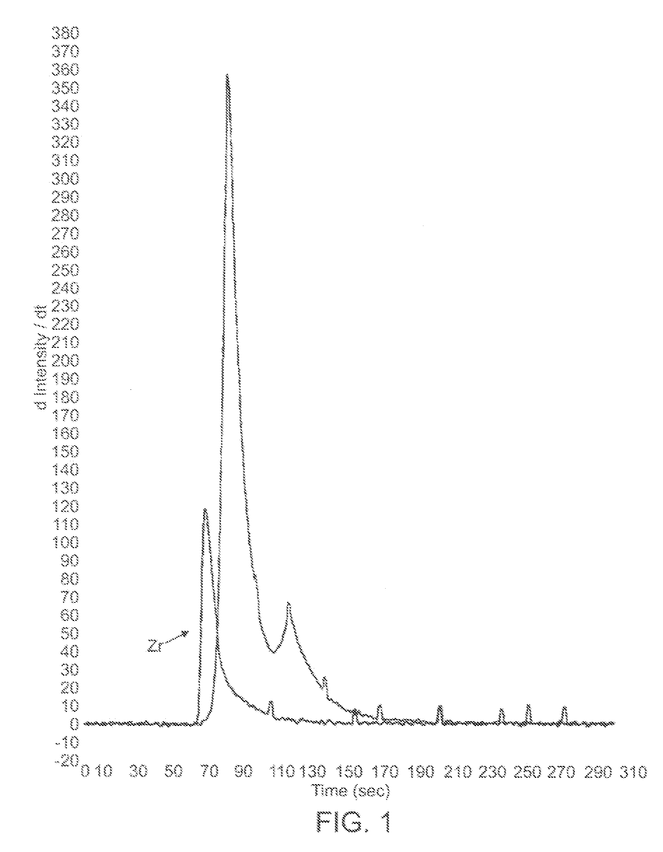

FIG. 2 is the ²⁷Al NMR of acid activated aluminum-zirconium-glycine powder at glycine/(Al+Zr) ratio of 0.10 according to experiment number 26 herein below described.

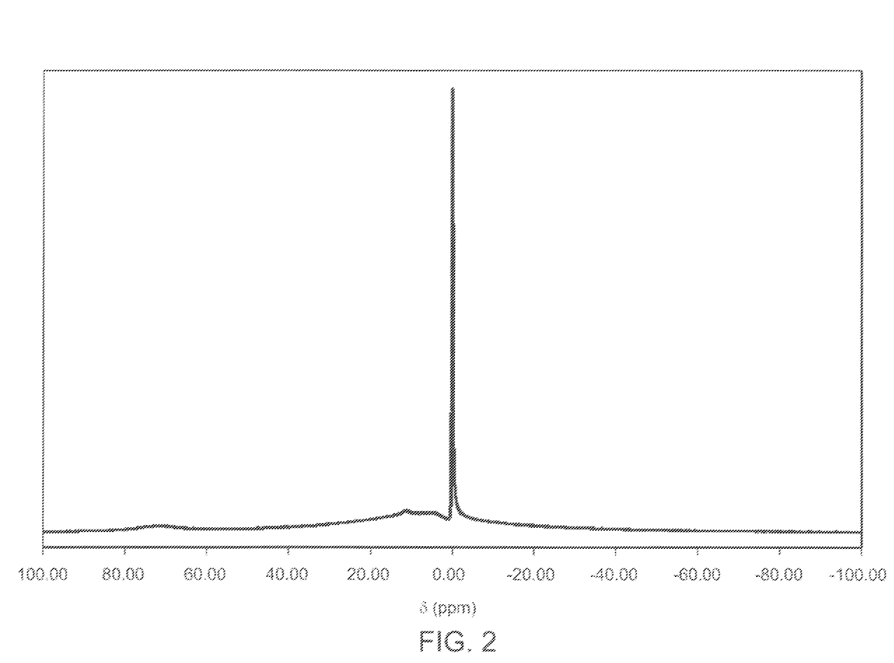

### DETAILED DESCRIPTION OF THE INVENTION

The present invention discloses enhanced efficacy aluminum-zirconium antiperspirant salt compositions comprising relatively low amino acid and relatively low zirconium and that are acid activated at metals to chloride (M/Cl) ratio of greater than 1.1. The compositions are prepared by the reaction of BAC solution, zirconium compounds, amino acid/or buffer and aluminum chloride.

The BAC solution for the present invention has the following empirical formula:

Al₂(OH)₆₋ₓClₓ

wherein 1.1≦x≦1.7. The BAC solutions can be prepared by both direct and indirect processes at 30%-50% by weight solution concentrations. The direct process relates to the direct reaction between AlCl3 or HCl aqueous solution with Al metal including Al powder and pellet and the indirect process corresponds to the reaction of ACH or BAC solution with AlCl3 or HCl.

The zirconium compound has the formula:

ZrO(OH)_{b}Cl_{2-b}

wherein 0≦b≦1 with ZrOCl₂ is the most preferred zirconium source. Zirconium Hydroxy Chloride (ZHC) can by prepared by mixing basic zirconium carbonate with HCl or zirconyl chloride aqueous solution at elevated temperatures. The ZHC solution may further be mixed with amino acid such as glycine/or buffer at temperatures of from RT to about reflux depending on the Cl/Zr ratios, i.e. at higher Cl/Zr ratio of 1.5 or above, the mixing can be carried out at RT and at lower ratio such as 1.2, the ZHC and glycine/or buffer solution can be heated to reflux. The ZHC glycinate/or buffer solutions are added to the BAC solution. Further, the order of addition is not critical and the aluminum-zirconium solutions can be dried to powders by appropriate means, such as spray drying. The ratios of Al/Cl of the BAC solutions and the Cl/Zr of the ZHC solutions can be varied depending on the M/Cl ratios of the final Al—Zr solutions. Preferably the BAC solutions have Al/Cl ratios of 1.3 to 1.8 and the ZHC solutions with Cl/Zr ratios of 1.2 to 2.

The Al/Zr ratio can be varied from about 3 to about 10, preferably from about 5 to about 10 and most preferably from about 8 to about 10. The aluminum-zirconium salts thus prepared have a M/Cl ratio of more than 1.1, preferably about 1.2.

A polyhydric alcohol can be added to either BAC or ZHC solutions to stabilize the aluminum-zirconium solutions especially at the molar ratio of amino acid/or buffer to Zr of less than 1, such as 0.5. A preferred polyhydric alcohol stabilizer is propylene glycol and glycerin.

Buffers useful in a practice of the invention include amino acids, glycine, alanine, lysine, DL-valine, arginine and L(−)prolinates, ornithine, citrulline, hydroxyproline, cysteine, threonine, serine, methionine, glutamic acid and the mixtures thereof with glycine being preferred. Other nitrogen materials containing acid groups such as betaine and betaine HCl as well as glycinates such as sodium, calcium and zinc glycinates may also be used as buffering agents.

It has been found by the present invention that an Al—Zr-Betaine solution is much more stable than the corresponding glycine containing AZG solution with respect to the physical stability of the solution. The quantity of the butter is controlled such that the molar ratio of the buffer to zirconium is about 0.5 to 1. Depending on the amount of zirconium, if the Al—Zr is a tetra salt, the molar ratio of buffer to zirconium must be controlled to be less than 1 such as 0.5, if the Al—Zr is an octa salt, the ratio should be about 1. The mole ratio of amino acid/or buffer to total metals (Al+Zr) should be about 0.05 to about 0.2, most preferably from about 0.08 to about 0.15.

According to the present invention, the BAC amino acid/or buffer solution of enhanced efficacy and increased stability has Al/Cl ratio of about 1.3 to 1.5 with the molar ratio of amino acid/or buffer to Al of about 0.05 to about 0.15, most preferably about 0.06 to 0.12. It has been found according to present invention that the BAC solutions at lower Al/Cl ratio such as below 1.1 and in the presence of less amino acid or other buffer, such as with the molar ratio of amino acid to Al less than 0.05 are very acidic and may cause skin irritation; however if the ratio is too high such as above 0.15, the solutions are unstable and will gel quickly. If the BAC solutions have higher Al/Cl ratio such as above 1.5, the basicity of the solutions is high enough that does not require the presence of amino acid for pH adjustment.

We have found that the presence of amino acid/or buffer such as glycine reduces the effective amount of Al monomers in BAC solutions dramatically and the resultant BAC-Glycine solutions gelled quickly especially at lower Al/Cl ratios as demonstrated in Experiments 1 and 2. The higher the amount of glycine, the lower the Al/Cl ratio of the BAC solution, the quicker the solution gels. As described in the prior art, the addition of zirconium solution to the Al containing solution depolymerizes the aluminum species, actually 50% activated Al—Zr-Glycine solution (made from the addition of ZHC glycinate solution to the activated ACH solution) has the similar Al compositions as 10% ASCH (aluminum sesqui solution, or otherwise BAC solution) by ²⁷Al NMR. About 40% to 50% glycine was estimated to be complexed in AZG solution. Not wishing to be bound with theories, it is believed that the free glycine will interact with small aluminum species such as Al monomers, results in the gelling of the Al—Zr-glycine solution especially at lower M/Cl ratio, which further reduces the efficacy of the corresponding antiperspirant salts because of the reduction of the amount of Al monomers. Such a practice of the present invention, has been observed that when the nitrogen containing carboxylic acid, such as betaine, is present in the BAC solution, the resultant BAC betaine solution does not gel even though the Al monomers are reduced. It is believed also that the interaction between glycine and Al-monomer species is through the hydrogen atoms of the amino group, which is absent in the case of betaine, and the interaction aluminum species and betaine is more ionic in nature.

The characterization of aluminum and zirconium species in aluminum-zirconium, antiperspirant salts are determined by the following methods.

### HPLC (Size Exclusion Column)

The degree of the polymerization of aluminum complexes is determined by Size Exclusion Chromatography (SEC) operated via a High Performance Liquid Chromatograph (HPLC) instrument. In this technique, the highest molecular weight Al species are eluted first and are designated as Band I or otherwise called peaks 1 & 2. Band II (or peak 3) and Band III (or peak 4) are due to intermediate molecular weight Al complexes. Band IV or peak 5 is due to the lowest molecular weight Al complexes, including monomers and dimmers. The relative area of one or more peaks is determined in order to characterize the distribution of polymeric species in aluminum complexes formed. The aluminum and aluminum-zirconium antiperspirant salts of the present invention have SEC-HPLC Band IV peak area of at least 20%.

### Nuclear Magnetic Resonance Spectroscopy

²⁷Al Nuclear Magnetic Resonance (NMR) is selected to identity the structures of different aluminum species in the aluminum and aluminum-zirconium antiperspirant salts of enhanced efficacy. The antiperspirant salt in solution form is measured as is and the powder is dissolved in deuteriated water to form a 10% by weight solution just before the measurement. Data were collected using a Varian Inova 400 instrument at 104.2 MHz.

Generally a sharp peak at chemical shift of about 0 ppm by ²⁷Al NMR indicates the presence of monomeric octahedral Al species. A peak at 4-5 ppm generally relates to octahedral Al dimer. A sharp peak at about 63 ppm from a tetrahedral aluminum species, indicates the presence Al₁₃ species which contains one tetrahedral Al center. The other 12 octahedral Al centers in this compound have peaks that are too broad to be detected. Larger aluminum species (greater than 41 mer) usually show chemical shifts that are too broad to be detected. The aluminum-zirconium antiperspirant salts of enhanced efficacy having low zirconium and glycine demonstrate higher amount of Al monomers at 0 ppm.

### Inductively Coupled Plasma Spectroscopy (ICP) SEC-HPLC

The present invention employs ICP-SEC-HPLC analysis to study the polymerization of zirconium species and the distribution of the aluminum polymers. The size exclusion column operated by HPLC has been used to separate the aluminum and zirconium polymers by size. The separated species are sent to the ICP instead of a refractive index (RI) detector as in regular HPLC. The invention will be further illustrated by the following examples. In the examples, parts are by weight unless otherwise specified.

### EXAMPLE 1

### Interaction of BAC Solutions with Glycine at Different Al/Cl Ratios

Five BAC and one ACH solutions (EXPERIMENT NO.6) were prepared to have the ratios of Al/Cl of from 1.19 to 1.92 by direct process. Glycine was added to the above solutions at a molar ratio of Glycine/Al of about 0.28. Table I lists the results.

TABLE I

| BAC Solutions | | | | | BAC-Glycine Solutions | | |
|---|---|---|---|---|---|---|---|
| Experiment | | Al/Cl | | ²⁷Al NMR | Solution | | ²⁷Al NMR |
| Number | % Al | Ratio | pH(as is) | % @ 0 ppm | Stability | pH(as is) | % @ 0 ppm |
| 1 | 10.26 | 1.19 | 2.57 | 15.68 | gel < 10 day | 2.87 | — |
| 2 | 10.08 | 1.28 | 2.75 | 14.21 | gel/11 days | 3.07 | 4.82 |
| 3 | 10.15 | 1.38 | 2.87 | 11.62 | gel/2 weeks | 3.20 | — |
| 4 | 10.90 | 1.48 | 2.91 | 9.49 | gel/3 weeks | 3.29 | 1.62 |
| 5 | 11.50 | 1.65 | 3.06 | 4.74 | gel/1.5 MO | 3.71 | — |
| 6 | 10.40 | 1.92 | 3.77 | 0.61 | gel/2.5 MO | 4.33 | — |

The data indicate more Al monomers are present in the BAC solutions at lower Al/Cl, however the BAC solutions gel up quickly when glycine was used to buffer the solution pH. Further, at similar glycine level the BAC solution gels faster at lower Al/Cl ratio even though the pH of the solution is lower, indicating stronger interactions occurred between aluminum species and glycine for the more depolymerized BAC solutions. The interaction between aluminum species in the BAC solutions and glycine is further demonstrated by ²⁷Al NMR by the reduction of the amount of Al monomers at 0 ppm as demonstrated in Experiment Number 2 where % Al monomers at 0 ppm has been reduced dramatically from about 14.21 to about 4.82.

### EXAMPLE 2

### Interactions of BAC Solutions with Different Amount of Glycine

Different amounts of glycine were mixed with BAC solutions at Al/Cl ratio of 1.28 and the results are listed in Table II.

TABLE II

| Experiment | Glycine/Al | | | ²⁷Al NMR | |
|---|---|---|---|---|---|
| Number | Ratio | pH (as is) | % Band IV | % @ 0 ppm | Solution Stability |
| 2 | 0 | 2.57 | 36.7 | 14.21 | liquid/1 year |
| 2 | 0.28 | 2.75 | — | 4.82 | gel/11 days |
| 7 | 0.14 | 3.00 | 38.1 | 6.71 | gel/2.5 MO |
| 8 | 0.12 | 3.00 | 37.8 | 7.19 | gel/4.5 MO |
| 9 | 0.098 | 2.99 | 33.5 | 7.68 | 450 cps/1.5 years |
| 10 | 0.098* | 2.95 | 37.6 | 6.64 | 10 cps/1.5 years |

*betaine

The data show that the interaction between aluminum species in the BAC solutions and glycine decreases the amount of Al monomer species. The higher the amount of glycine, the lower the amount of Al monomers, and the less stable the solution becomes. Actually the presence of higher amount of glycine does not seem to change the pH of the BAC solutions dramatically, however it reduces the stability of the solution greatly. It is observed that the interactions between Al species and glycine can hardly be detected by SEC-HPLC, i.e. HPLC Band IV of the BAC solution does not change or even increases slightly upon the addition of glycine or betaine.

### EXAMPLE 3

### Comparison of the Interaction of BAC Solutions with Glycine and Betaine

In the experiments 11 and 12, glycine and betaine were added to the 40% BAC solutions at glycine (or betaine) to Al molar ratios of about 0.12 with Al/Cl ratios of 1.38 and 1.48, respectively. In Experiment 13, a 30% by weight BAC solution was prepared by direct process at Al/Cl ratio of 1.47, glycine and betaine were added respectively to the BAC solution at hot with glycine (or betaine)/Al molar ratio of about 0.12. The stability of the solutions is monitored and the results are summarized in Table III.

TABLE III

| BAC-Glycine Solution | | | | BAC-Betaine Solution | |
|---|---|---|---|---|---|
| Experiment | Al/Cl | pH | | pH | |
| Number | Ratio | (as is) | Stability | (as is) | Stability |
| 11 | 1.38 | 3.10 | gel/5 MO | 3.10 | 60 cps/1.5 years |
| 12 | 1.48 | 3.16 | gel/5 MO | 3.16 | 64 cps/1.5 years |
| 13 | 1.47 | 3.66 | gel/8 days | 3.73 | 6 cps/1.5 years |

From the results it is believed that the interaction between the glycine and Al species is through the amino group of glycine with Al species to form the gel. For betaine, the absence of amino group prevents the gelling of BAC-betaine solutions, even though the interaction between betaine and Al monomers still exist as demonstrated in Experiment 10 by ²⁷Al NMR.

### EXAMPLE 4

### Comparison of Al—Zr Tatra and Octa Salts at Glycine/Zr Molar Ratios at 1 and 0.5

The Al—Zr salts were prepared by the mixing of 45% BAC solutions with Al/Cl ratios of 1.63 and 1.31 with ZrOCl₂ glycinate solutions at different glycine/Zr molar ratios. The solutions were spray dried to powders.

TABLE IV

| Experiment | | | Glycine/Zr | ²⁷Al NMR |
|---|---|---|---|---|
| Number | Al/Zr Ratio | M/Cl Ratio | Ratio | % @ 0 ppm |
| 14 | 5 | 1.2 | 1 | 19.42 |
| 15 | 5 | 1.2 | 0.5 | 25.56 |
| 16 | 8 | 1.3 | 1 | 16.42 |
| 17 | 8 | 1.3 | 0.5 | 15.45 |
| 18 | 5 | 1 | 1 | 20.06 |
| 19 | 5 | 1 | 0.5 | 24.66 |
| 20 | 8 | 1.1 | 1 | 18.99 |
| 21 | 8 | 1.1 | 0.5 | 22.08 |

The data indicate that more aluminum monomers were formed in the presence of less amount of glycine. The effect of the amount of glycine on the amount of aluminum monomer is not as dramatic at higher Al/Zr ratio such as 8 as at lower ratio such as 5 at M/Cl ratio above 1.2.

### EXAMPLE 5

### Comparison of Activated Al—Zr-Tetrachlorohydrex Glycine Salts Having Low and High Level of Glycine

A 12% by weight BAC solution with Al/Cl ratio of about 1.7 that was refluxed for 2 hours were mixed with ZHC glycinate and ZHC glycinate propylene glycol solutions. respectively at RT. The solutions were spry dried to give two powders and the analysis is as follows.

TABLE V

| Experiment | Al/Zr | M/Cl | | Glycine/M | ²⁷Al NMR |
|---|---|---|---|---|---|
| Number | Ratio | Ratio | % Glycine | Ratio | % @ 0 ppm |
| 22 | 3.3 | 1.18 | 14.66 | 0.32 | 7.95 |
| 23 | 3.6 | 1.25 | 5/4.1% PG | 0.10 | 15.55 |

The data again demonstrate the effect of higher glycine on the reduction of the Al monomer species, which further reduced the efficacy of antiperspirant salts.

### EXAMPLE 6

### Comparison of Al—Zr-Octa- and Pentachlorohydrex Glycine Salts with Activated Al—Zr-Tetrachlorohydrex Glycine Salt

Al—Zr-glycine octa salt solutions were prepared by mixing 45% BAC solutions, AlCl₃ (32⁰Be) and ZrOCl₂ glycinate solutions and spray drying the final solutions to powders. The penta salt was made similarly except no AlCl₃ was added.

TABLE VI

| Experiment | Al/Zr | M/Cl | | Glycine/M | ²⁷ Al NMR |
|---|---|---|---|---|---|
| Number | Ratio | Ratio | % Glycine | Ratio | % @ 0 ppm |
| 24 | 8.1 | 1.22 | 6.25 | 0.11 | 21.13 |
| 25 | 8.0 | 1.56 | 6.85 | 0.11 | 8.43 |
| 22 | 3.3 | 1.18 | 14.66 | 0.32 | 7.95 |

As can be seen from the table that the amount of Al monomers in the octa salt is much higher than that present in the activated Al—Zr tetra salt at similar M/Cl ratio, although the tetra salt has much more zirconium, and surprisingly, even the penta salt has equivalent amount of Al monomers as the tetra salt. Further the addition of acid, such as AlCl₃, increases the amount of aluminum monomer species, which enhances the efficacy of the aluminum-zirconium-octa salt. The solution of the octa salt is stable for at least a year at RT.

### EXAMPLE 7

### Comparison of Acid Activated Al—Zr-Glycine Containing Low Glycine and Zirconium with Heat Activated Al—Zr-Glycine Glycine Salt

50% Reach 301 (a product from Reheis with Al/Cl ratio of 1.7) solution, AlCl₃ and zirconium oxychloride glycinate solutions were mixed and spray dried to form an acid activated Al—Zr-glycine powder with low glycine and zirconium as experiment number 26. In experiment number 27, ACH solution was first prepared by diluting the solution to about 15% by weight, followed by heating the solution to form an AACH solution with high Band III/II ratio, cooling the solution, and finally mixing the solution with zirconium hydroxychloride glycinate solution and quickly spray drying the solution to form a heat activated Al—Zr-glycine powder with high glycine and zirconium. The results are shown in Table VII.

TABLE VII

| | | | | HPLC Band |
|---|---|---|---|---|
| Experiment | | | Glycine/M | III/II |
| Number | Al/Zr Ratio | M/Cl Ratio | Ratio | Ratio |
| 26 | 9.04 | 1.24 | 0.1 | 0.25 |
| 27 | 4.21 | 1.26 | 0.2 | 1.20 |

Experiment number 26 showed significant better efficacy than experiment number 27 when formulated in stick formulation.

What is claimed:

1. A method for preparing enhanced efficacy antiperspirant compositions selected from a basic aluminum chloride (BAC) salts having low buffer content and aluminum-zirconium salts having low buffer and low zirconium comprising, respectively;
(i) reacting an acid activated BAC solution having an Al/Cl ratio between about 1.3 to about 1.5 with a buffer at a buffer to aluminum molar ratio of from about 0.05 to about 0.15 to yield aluminum antiperspirant solutions having the following empirical formula:
Al₂(OH)₆₋ₓClₓ(AA)_{y}
wherein x has the value: 1.3≦x≦1.6, AA is a buffer system which includes an amino acid and nitrogen containing base, y has the value: 0.1≦y≦0.4; and
(ii) reacting BAC solution having concentration of between about 30 percent and 50 percent and an Al/Cl ratio below about 1.8 and an HPLC Band I of less than about 5 percent and a zirconium compound of the formula;
ZrO(OH)_{b}Cl_{2-b}
wherein 0≦b≦1, a buffer in amounts of less than 5 percent by weight and an acid to yield aluminum-zirconium antiperspirant salts having the following empirical formula:
AlₙZr(OH)₍₃ₙ₊₄₋ₓ₎Clₓ(AA)_{y} Rₚ
wherein n is from 3 to 10, preferably 6 to 10;
x is from 3 to 9, calculated from M/Cl ratio, preferably with M/Cl ratio greater than 1:1;
AA is a buffer, which includes an amino acid and a nitrogen containing base, y is from 0.055 to 0.22 calculated from y/M molar ratio, which is from 0.05 to 0.2, preferably from 0.05 to 0.15;
R is an organic solvent having at least two carbon atoms and at least one hydroxyl group and “p” has a value from zero to 1.5.

2. A method for preparing enhanced efficacy selected from a basic aluminum chloride (BAC) antiperspirant compositions having low buffer content comprising;
(i) reacting an acid activated BAC solution having an Al/Cl ratio between about 1.3 to about 1.5 with a buffer at a buffer to aluminum molar ratio of from about 0.05 to about 0.15 to yield aluminum antiperspirant solutions having the following empirical formula:
Al₂(OH)₆₋ₓClₓ(AA)_{y}
wherein x has the value; 1.3≦x≦1.6, AA is a buffer system which includes an amino acid and nitrogen containing base, y has the value: 0.1≦y≦0.4.

3. A method of preparing enhanced efficacy basic aluminum chloride (BAC) salts having low buffer content and aluminum-zirconium antiperspirant compositions having low buffer and low zirconium comprising:
(i) reacting BAC solution having concentration of between about 30 percent and 50 percent and an Al/Cl ratio below about 1.8 and an HPLC Band I of less than about 5 percent and a zirconium compound of the formula:
ZrO(OH)_{b}Cl_{2-b}
wherein 0≦b≦1, a buffer in amounts of less than 5 percent by weight and an acid to yield aluminum-zirconium antiperspirant salts having the following empirical formula:
AlₙZr(OH)₍₃ₙ₊₄₋ₓ₎Clₓ(AA)_{y} Rₚ
wherein n is from 3 to 10, preferably 6 to 10; x is from 3 to 9, calculated from M/Cl ratio, of at least 1:1; AA is a buffer, which includes an amino acid and a nitrogen containing
base, y is from 0.055 to 0.22 calculated from y/M molar ratio, which is from 0.05 to 0.2, preferably from 0.05 to 0.15;
R is an organic solvent having at least two carbon atoms and at least one hydroxyl group and “p” has a value from zero to 1.5.

4. The method of claim 1 wherein the buffer is an amino acid.

5. The method of claim 4 wherein the amino acid is glycine.

6. The method of claim 1 wherein the buffer is betaine.

7. The method of claim 2 wherein the BAC solution is a reaction product formed by a direct reaction between an acid selected from AlCl₃ and HCl and aluminum metal powder as selected.

8. The method of claim 2 wherein the BAC is derived from the indirect process of reacting an aluminum solution selected from ACH and aluminum sesquichlorohydrate with an acidic reagent selected from HCl and AlCl₃.

9. The method of claim 1 wherein the prepared composition is dried.

10. The method of claim 2 wherein the atomic ration of aluminum to chloride of the BAC solution is below 1.8.

11. The method of claim 3 wherein the zirconium compound is zirconium oxychloride.

12. The method of claim 3 wherein the atomic ratio of aluminum to zirconium is above 4.

13. The method of claim 12 wherein the atomic ration of aluminum to zirconium is above 6.

14. The method of claim 13 wherein the atomic ratio of aluminum to zirconium is above 8.

15. The method of claim 3 wherein the molar ratio of metals (Al+Zr) to chloride is 1.2.

16. The method of claim 1 wherein the amino acid/or buffer to metals is from 0.05 to 0.2.

17. The method of claim 16 wherein the amino acid or buffer to metals is from 0.08 to 0.15.

18. The products produced by the method of claim 1.

19. The products produced by the method of claim 9.

20. The method of claim 2 wherein the BAC solution is prepared by a process selected from either direct or indirect process at a solution concentration of 30%-50% by weight.

21. The method of claim 20 wherein the products are dried.

22. The method of claim 20 wherein the BAC solution has atomic ratio of aluminum to chloride is about 1.3 to about 1.5.

23. The method of claim 20 wherein the BAC is derived from the direct reaction between an aqueous acidic solution selected from HCl and AlCl₃ with aluminum metal.

24. The method of claim 20 wherein the BAC is derived from the indirect process of reacting an aluminum solution selected from ACH or aluminum sesquichlorohydrate with an acidic reagent selected from HCl and AlCl₃.

25. The method of claim 20 wherein the molar ratio of amino acid/or buffer to aluminum is about 0.05 to about 0.15.

26. The method of claim 20 wherein the amino acid is glycine.

27. The method of claim 20 wherein the buffer is betaine.

28. The products produced by the method of claim 20.

29. The products produced by the method of claim 21.
